(19) United States
(12) Patent Application Publication
(10) Pub. No.: US 2026/0034043 A1
(43) Pub. Date: Feb. 5, 2026

(54) NAIL COMPOSITIONS INCLUDING SULFUR-CONTAINING AGENTS AND USES THEREOF

(71) Applicant: Liqwd, Inc., Santa Barbara, CA (US)

(72) Inventors: Craig Hawker, Santa Barbara, CA (US); Christopher M. Bates, Santa Barbara, CA (US); Javier Read de Alaniz, Santa Barbara, CA (US); Morgan Bates, Santa Barbara, CA (US); Kaitlin Albanese, Santa Barbara, CA (US)

(21) Appl. No.: 19/284,028

(22) Filed: Jul. 29, 2025

Related U.S. Application Data
(60) Provisional application No. 63/677,372, filed on Jul. 30, 2024.

(51) Int. Cl. A61K 8/49; A61Q 3/02

(52) U.S. Cl. CPC A61K 8/4986; A61Q 3/02; A61K 2800/43; A61K 2800/81

(57) ABSTRACT

Nail compositions including sulfur-containing active agents which are capable of reacting and forming a nail coating, as well as methods of using the nail compositions are described herein. The nail compositions can be, for instance, in the form of nail strengtheners, nail primers, nail polishes, nail lacquers, gel nails, or dip nails.

### CROSS-REFERENCED TO RELATED APPLICATIONS

This application claims the benefit of and priority to U.S. Provisional Application No. 63/677,372, filed Jul. 30, 2024, which is hereby incorporated herein by reference in its entirety.

### FIELD OF THE INVENTION

The disclosed invention is generally in the field of cosmetics, particularly in the field of compositions and treatments for nails.

### BACKGROUND OF THE INVENTION

Nails serve as protective plates located at the tip of digits such as fingernails and toenails. The top component of the nail exposed to air, known as the nail plate, is prone to damage such as cracking, tearing, thinning, and weakening that can cause painful sensitivity to touch, temperature, gripping, grasping, or other common situations.

Sometimes damage to the nail plate occurs through everyday activities. Another source of damage involves the use of nail coating products that are commonly applied to the nail plate as a fashion statement, for decorative purposes, or as part of beauty regimens. Examples of beauty products that can damage the nail plate during application, wear, and/or removal include nail polish (e.g., lacquers, varnishes, and enamels), gels, dips, nail enhancements, and artificial nails. Nail polishes typically include various non-volatile molecules or solid components that are dissolved and/or suspended in non-reactive solvents. Upon application and drying, the non-volatile molecules or solid components deposit on the nail surface as a clear, translucent, or colored film. Because nail polishes lack mechanical robustness, they are easily scratched and often chip or peel from the natural nail plate approximately one to five days after application. Such chipping, peeling, and scratching can cause damage to the underlying natural nail plate. Additionally, because nail polishes form a film by simple evaporation of non-reactive solvents, they can be removed by rubbing or soaking using the same (or similar) non-reactive solvents. Repeated application and removal of nail polishes in this way exposes the nail plate to solvents and chemical agents that can swell and de-swell the nail plate, which is another potential source of damage.

Commercially available gel manicures are popular nail products that last for up to approximately two weeks without chipping or significant damage. In gel manicures, a base coat, color coat(s), and/or a top coat are applied layer by layer and “cured” underneath a UV light before applying the next layer. The UV light causes a photoinitiator in each coat or layer to create radicals that induce polymerization and crosslinking of the constituent acrylate, methacrylate, acrylamide, and/or methacrylamide monomers. While gel products often last longer than nail polish because such crosslinking results in more mechanically robust coatings, this longevity makes the gel manicure much more difficult to remove. So-called “hard” gels usually require aggressive mechanical abrasion to remove that can cause significant damage to the underlying nail plate. “Soft” gels sacrifice some (but not all) crosslinking to make the coating more prone to swelling with solvents such as 100% acetone, which facilitates removal by less aggressive mechanical removal; however, soaking with these solvents often requires long contact times (upwards of 10 to 30 minutes) that cause discomfort, swelling of the nail plate, dehydration of the nail plate, and/or associated cracking and/or weakening of the nail plate, as well as damage to the surrounding skin. There are also reported cases of bleeding after gels start peeling, either during use or removal, or rough surfaces/edges catching or snagging leading to nail ripping and further damage and discomfort. Both hard and soft gels are therefore another source of damage to the nail plate that is particularly prevalent given their popularity in today's society.

A third class of commercially available nail product known as “dip” involves a process whereby a thin coating of liquid cyanoacrylate is applied, followed quickly by dipping the wet nail into a solid powder that also forms a thin layer on top of the cyanoacrylate. This process can be repeated multiple times to build up a stack of alternating layers, optionally followed by other layers that may contain activators to accelerate the solidification of underlying cyanoacrylates, and a top coat to improve final sheen. This cyanoacrylate chemistry is analogous to super glue. Thus, dip products are long lasting and even more difficult to remove than hard gels. Removing dip requires extremely aggressive mechanical abrasion that often causes significant damage to the underlying nail plate. Sometimes, other nail products, such as wraps, tip adhesives, nail enhancements and artificial nails are attached to the nail plate using cyanoacrylate chemistry because it is such an effective adhesive. Like dip, the polymerized cyanoacrylate is difficult, if not impossible, to remove from the nail plate without causing damage to the user.

There are few convenient options that strengthen the natural nail bed for people or animals, either to prevent damage before it occurs or protect a damaged nail from further injury. Although the aforementioned nail polish, gel, or dip coatings act as a physical barrier against external damage to the underlying nail bed, as described above, their application, use, and/or removal often damage the nail bed. Thus, use of such nail treatments (and their resulting removal) creates a difficult-to-escape cycle.

For already damaged nails, aldehyde treatments with formaldehyde, citral, and citronellal formulations are known to harden nails by forming bonds between protein residues in keratin, but these have well-known drawbacks related to health and safety. Other commercially available nail strengtheners also contain unsafe ingredients, including hydrazines, divinyl sulfone, and dithiopyridine.

A conceptually similar mechanism of crosslinking protein residues in keratin that involves the use of Michael acceptors such as bismaleimides and related derivatives requires repeated application and penetration of the formulation deep into the nail plate that can be a major challenge to achieve in practice.

There remains a need for nail compositions which can address and overcome one or more of the challenges associated with commercially available nail compositions for strengthening nails and/or repairing damaged nails and/or serving as a layer attached directly to the nail onto which other nail enhancements can be applied.

Accordingly, it is an object of the invention to provide improved nail compositions for strengthening nails and/or repairing damaged nails and/or priming nails.

It is a further object of the invention to provide methods of using such improved nail compositions for strengthening nails and/or repairing damaged nails and/or priming nails.

### BRIEF SUMMARY OF THE INVENTION

Described herein are nail compositions containing sulfur-containing agents and their uses in nail care applications.

In one non-limiting instance, such a nail composition includes:
an active agent having a molecular structure comprising at least one cyclic ring containing a disulfide or polysulfide bond; and
a cosmetically acceptable carrier;
wherein the active agent is capable of undergoing a reaction, optionally a polymerization reaction, when exposed to a stimulus, such as light or heat;
wherein the concentration of active agent is sufficient to form a nail coating on a nail surface following application to the nail surface and exposure to the stimulus; and
wherein the nail coating is adherent to the nail surface.

In certain instances, the active agent is α-lipoic acid, an ester of α-lipoic acid, an anhydride of α-lipoic acid, a salt of α-lipoic acid, or an amide of α-lipoic acid. In some other instances, the active agent is 1,2-dithiolane (such as lipoic acid), asparagusic acid, 1,2,3-trithiolane, S₈, 1,2-dithiane, 1,2-dithepane, or combinations thereof. Other active agents of Formulae I-IV and multifunctional active agents are also described below.

In some instances, the nail compositions include monomeric active agents, polymers formed therefrom, or combinations thereof. In some instances, the nail compositions include an active agent that is a monomer capable of being polymerized. In certain other instances, the nail compositions include an active agent that has been at least partially or fully polymerized without the need for exposure to an external stimulus, where the active agent includes disulfide or polysulfide bonds.

In some instances, the nail compositions further include one or more co-reactive monomers capable of undergoing copolymerization or condensation with the active agent.

Without limitation, the nail compositions can include one or more cosmetically acceptable excipients, such as: humectants, emollients, oils, moisturizers, vitamins, and/or fragrances. The nail compositions may include one or more pigments/dyes to provide a color to the nail coating formed. In some instances, the nail compositions exclude such pigments and/or dyes.

The nail compositions can include one or more solvents, carriers, and/or diluents, which are typically liquids, such as water (deionized, distilled, or purified), alcohols, fatty alcohols, polyols, low molecular weight polymers (polymers with weight average molecular weights below about 10,000 g/mol, such as dimethicone, poly(ethylene glycol), high molecular weight polymers (polymers with weight average molecular weights above about 10,000 g/mol, such as poly(methyl methacrylate), acrylate copolymers, and the like), or mixtures thereof. The solvent and/or carrier are selected to evaporate when the nail composition is applied to the nail surface.

In some instances, the nail compositions further include one or more photochemical and/or thermal initiators.

In some instances, the nail compositions further include at least one stabilizing agent(s), thiol-capping agent(s), and/or UV absorbing agent(s). In certain instances, the nail compositions further include one or more additives that increase abrasion resistance or scratch resistance of the formed nail coating.

The nail compositions described herein can be used in various applications in the nail care industry. In one non-limiting instance, a method of forming a coating on a nail with the nail compositions includes the steps of:
(a) applying the nail composition to a subject's nail; and
(b) allowing the cosmetically acceptable carrier to evaporate to form a nail coating, optionally an initial coating, on the nail of the subject.

Optionally, the above method further includes a step of exposing the nail composition to a stimulus, such as light or heat, prior to, during, or following evaporation of the cosmetically acceptable carrier, where the stimulus is sufficient to induce a reaction of at least some of the active agent, for example, to undergo at least partial polymerization of at least a portion of the active agent to form a polymer or coating containing disulfide or polysulfide bonds therein.

In some instances, the method forms a nail coating that provides a sheen or gloss on the nail, as compared to the nail prior to formation of the nail coating thereon. Such an improved sheen or gloss can in some instances be due to an increase in the refractive index of the coating compared to the natural nail plate or the generation of a smoother surface. In some instances, the method forms a nail coating that strengthens, protects, provides scratch resistance, repairs, and/or hardens the subject's nail, as compared to the nail prior to formation of the nail coating thereon. In some instances, the method forms a nail coating that increases resistance to wear and/or abrasion, and/or tearing, increases robustness, improves or changes the adhesion of any additional coatings applied after the nail coating, and/or improves the look and/or feel of the subject's nail, as compared to the nail prior to application of the nail composition thereon. In some instances, the method forms a nail coating which provides an improvement in look, feel, and/or robustness, as assessed by visual or tactile inspection and may comprise a reduction of ridges and the filling in of defects present in the subject's nail prior to formation of the nail coating thereon.

The nail compositions described herein can be packaged together in any suitable combination as a kit useful for the disclosed methods. For example, the kits are provided for nail care applications, such as use as a nail strengthener, a nail primer, a nail protector, a nail polish, a nail gel, a nail extension, or a nail dip.

### BRIEF DESCRIPTION OF THE DRAWINGS

The accompanying drawings illustrate several embodiments of the disclosed compositions and methods thereof which together with the description, serve to explain the principles of the disclosed methods and compositions.

FIGS. 1A and 1B are two photographs comparing damaged nails before (FIG. 1A) and after (FIG. 1B) application of a lipoic-acid nail coating, as discussed in the Examples.

### DETAILED DESCRIPTION OF THE INVENTION

Nail compositions containing sulfur-containing agents and their uses in nail care applications are described herein.

### I. Definitions

The term “cosmetically acceptable” means compatible with the keratin (i.e., nail) and skin tissues and/or its integuments, which has an acceptable color, odor, and feel, and which does not cause any unacceptable discomfort (stinging or tautness) liable to discourage a subject or consumer from using the composition.

The term “coating” refers to a deposit formed on at least a portion of a surface of a keratin material, such as a nail surface, where the deposit adheres to the keratin material and is resistant to chipping and/or breakage for a given period of time, when exposed to everyday conditions and activities (such as washing hands with hand and dish soap, showering or bathing, touching and brushing against cloth, wood, metal and plastic surfaces, such as clothing, doors, tables, etc.).

The term “volatile” refers to solvent(s) which are capable of evaporating on contact with the nail surface in less than one hour, at room temperature and atmospheric pressure.

“Carboxylic acid”, as used in here refers to the group —COOH. Unless specified otherwise the term carboxylic acid embraces both the free acid and carboxylate salt.

“Alkyl”, as used herein, refers to saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain. C₃-C₃₀ for branched chain), more preferably 20 or fewer carbon atoms, more preferably 12 or fewer carbon atoms, and most preferably 8 or fewer carbon atoms. In some embodiments, the chain has 1-6 carbons. Likewise, preferred cycloalkyls have from 3-10 carbon atoms in their ring structure, and more preferably have 5, 6 or 7 carbons in the ring structure. The ranges provided above are inclusive of all values between the minimum value and the maximum value. The term “alkyl” includes both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, ether, ester, carbonyl (such as a carboxyl, alkoxycarbonyl, formyl, or an acyl), thiocarbonyl (such as a thioester, thionoester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, or an aromatic or heteroaromatic moiety.

Unless the number of carbons is otherwise specified, “lower alkyl” as used herein means an alkyl group, as defined above, but having from one to ten carbons, more preferably from one to six carbon atoms, in its backbone structure. Likewise, “lower alkenyl” and “lower alkynyl” have similar chain lengths. Preferred alkyl groups are lower alkyls. The alkyl groups may also contain one or more heteroatoms within the carbon backbone. Examples include oxygen, nitrogen, sulfur, and combinations thereof. In certain embodiments, the alkyl group contains between one and four heteroatoms.

“Alkenyl” and “Alkynyl”, as used herein, refer to unsaturated aliphatic groups containing one or more double or triple bonds analogous in length (e.g., C₂-C₃₀) and possible substitution to the alkyl groups described above.

“Aryl”, as used herein, refers to 5-, 6- and 7-membered aromatic rings. The ring may be a carbocyclic, heterocyclic, fused carbocyclic, fused heterocyclic, bicarbocyclic, or biheterocyclic ring system, optionally substituted as described above for alkyl. Broadly defined, “Ar”, as used herein, includes 5-, 6- and 7-membered single-ring aromatic groups that may include from zero to four heteroatoms. Examples include, but are not limited to, benzene, pyrrole, furan, thiophene, imidazole, oxazole, thiazole, triazole, pyrazole, pyridine, pyrazine, pyridazine and pyrimidine. Those aryl groups having heteroatoms in the ring structure may also be referred to as “heteroaryl”, “aryl heterocycles”, or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties. —CF₃, and —CN. The term “Ar” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic.

“Alkylaryl”, as used herein, refers to an alkyl group substituted with an aryl group (e.g., an aromatic or heteroaromatic group).

“Heterocycle” or “heterocyclic”, as used herein, refers to a group attached via a ring carbon or nitrogen of a monocyclic or bicyclic ring containing 3-10 ring atoms, and preferably from 5-6 ring atoms, containing carbon and one to four heteroatoms each selected from non-peroxide oxygen, sulfur, and N(Y) wherein Y is absent or is H, O, (C₁₋₄) alkyl, phenyl or benzyl, and optionally containing one or more double or triple bonds, and optionally substituted with one or more substituents. The term “heterocycle” also encompasses substituted and unsubstituted heteroaryl rings. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4H-carbazolyl, carbolinyl, chromanyl, chromenyl, cinnolinyl, decahydroquinolinyl, 2H,6H-1,5,2-dithiazinyl, dihydrofuro[2,3]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. “Heteroaryl”, as used herein, refers to a monocyclic aromatic ring containing five or six ring atoms containing carbon and 1, 2, 3, or 4 heteroatoms each selected from non-peroxide oxygen, sulfur, and N(Y) where Y is absent or is H, O, (C₁-C₂₀) alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benzo-derivative or one derived by fusing a propylene, trimethylene, or tetramethylene diradical thereto. Examples of heteroaryl include, but are not limited to, furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

“Halogen”, as used herein, refers to fluorine, chlorine, bromine, or iodine.

The term “substituted,” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aryloxy, substituted aryloxy, alkylthio, substituted alkylthio, phenylthio, substituted phenylthio, arylthio, substituted arylthio, cyano, isocyano, isocyanato, substituted isocyanato, isothiocyanato, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms, such as nitrogen, may have hydrogen substituents and/or any permissible substituents of organic compounds described herein that satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Polymer”, as used herein, refers to a molecule containing 5 or more monomer repeat units.

The term “effective period of time” or “effective amount of time” refers to the amount of time which is sufficient to cause a desired condition, effect, or outcome to occur. It is typically the minimum span of time required to effectively produce the intended result.

Numerical ranges disclosed herein disclose individually each possible number in such range, as well as any sub-ranges and combinations of sub-ranges encompassed therein. For example, a carbon range (e.g., C₁-C₁₀) is intended to disclose individually every possible carbon value and/or sub-range encompassed within. For example, a carbon length range of C₁-C₁₀ discloses C₁, C₂, C₃, C₄, C₅, C₆, C₇, C₈, C₉, and C₁₀, as well as discloses sub-ranges encompassed within, such as C₂-C₉, C₃-C₈, C₁-C₅, etc. Similarly, an integer value range of 1-10 discloses the individual values of 1, 2, 3, 4, 5, 6, 7, 8, and 10, as well as sub-ranges encompassed within. Further, a concentration range or weight percent range, such as from 1% to 2% by weight of the formulation discloses, the individual values and fractions thereof, such as 1%, 1.1%, 1.2%, 1.32%, 1.48% etc., as well as sub-ranges encompassed within.

### II. Nail Compositions

Various nail compositions are described herein which include sulfur-containing agents. Sulfur-containing agents generally have a molecular structure containing at least one cyclic ring containing a disulfide or polysulfide bond. One non-limiting example of a sulfur-containing agent is lipoic acid, also known as α-lipoic acid, alpha-lipoic acid (ALA) and thioctic acid.

Structurally, sulfur-containing agents, like lipoic acid, contain two sulfur atoms connected by a disulfide bond in a multi-membered ring structure, such as a dithiolane. Such agents can also include one or more substituents, such as alkyl side chain having one or more functional groups, such as a free carboxylic acid group, thereon. Such agents, like the 1,2-dithiolane ring of lipoic acid, are also capable of undergoing ring-opening polymerization to yield polymers, such as poly(disulfide)s.

Exemplary nail compositions containing a sulfur-containing agent are described below.

In one non-limiting instance, the nail composition includes:
an active agent having a molecular structure comprising at least one cyclic ring containing a disulfide or polysulfide bond; and
a cosmetically acceptable carrier;
wherein the active agent is capable of undergoing a reaction, optionally a polymerization reaction, when exposed to a stimulus, such as light or heat;
wherein the concentration of active agent is sufficient to form a nail coating on a nail surface following application to the nail surface and exposure to the stimulus; and
wherein the nail coating is adherent to the nail surface.

Such known reactions can include, but are not limited to, polymerization reactions, thiol coupling reactions, oxidative coupling reactions, thiol addition reactions, thiol click reactions, thiol-ene reactions, thiol-yne reactions, disulfide metathesis reactions, or combinations thereof. In some instances, the reaction is a polymerization reaction.

In some instances, the nail coating adheres to the nail surface and the nail coating remains on the nail surface when exposed to water, such as by dipping the nail in water, for at least about 30 minutes, 1 hour, 2 hours, 4 hours, 8 hours, 16 hours, 24 hours, 48 hours, 72 hours, or longer. In some instances, the nail coating adheres to the nail surface and the nail coating remains on the nail surface when exposed to an organic solvent, such as dipping in acetone, for at least about 5, 10, 15, 20, 25, or 30 minutes, or longer. In some instances, the deposit adheres to the nail surface and is resistant to chipping and/or breakage for a least about 5, 10, 15, 20, 25, or 30 minutes, or longer, when exposed to everyday conditions and activities (such as washing with hand and dish soap, showering or bathing, touching and brushing against cloth, wood, metal and plastic surfaces, such as clothing, doors, tables, etc.). In some instances, the resistance of the nail coating to solvents and such everyday conditions increases over time, for example, after 1 day, 2 days, 3 days, 5 days, 1 week, 2 weeks, 3 weeks, or longer.

Optionally, the active agent is present in a concentration of at least about 0.5, 0.6, 0.7, 0.8, 0.9 or 1 wt. %, or higher, of the total weight of the nail composition. In some instances, the active agent is present in a concentration in a range of about 0.5 to 50 wt. %, 0.6 to 45 wt. %, 0.7 to 40 wt. %, 0.8 to 35 wt. %, 0.9 to 30 wt. %, 1 to 25 wt. %, 5 to 20 wt. %, 10 to 20 wt. %, or 12 to 15 wt. %, of the total weight of the nail composition, as well as individual values or sub-ranges contained within the aforementioned ranges. In some other instances, the active agent is present in a concentration in a range of about 0.5 to 15 wt. %, 0.6 to 15 wt. %, 0.7 to 15 wt. %, 0.8 to 15 wt. %, 0.9 to 15 wt. %, or 1 to 15 wt. % of the total weight of the nail composition, as well as individual values or sub-ranges contained within the aforementioned ranges. For the aforementioned, the concentrations are prior to evaporation of any volatile cosmetically acceptable carrier(s) present in the nail composition and prior to undergoing any reactions, such as polymerization, of the active agent(s) in the nail composition.

In some instances, a coating is formed from the nail composition solely due to evaporation of all or substantially all of the volatile carrier(s) after being applied to the nail surface and can also be referred to as an “initial coating”. “Substantially all,” as used herein, refers to removal by evaporation of at least about 95%, 96%, 97%, 98%, 99%, 99.5%, or 99.9% of the volatile solvent from the nail coating.

In still other instances, when the initial coating is formed, the active agent can be present in a concentration ranging from about 1 to 100 wt. %, 3 to 100 wt. %, 5 to 100 wt. %, 10 to 100 wt. %, 15 to 100 wt. %, 20 to 100 wt. %, 25 to 100 wt. %, as well as individual values or sub-ranges of the aforementioned ranges. In some other instances, when the initial coating is formed, the active agent can be present in a concentration ranging from about 1 to 99.9 wt. %, 3 to 99.9 wt. %, 5 to 99.9 wt. %, 10 to 99.9 wt. %, 15 to 99.9 wt. %, 20 to 99.9 wt. %, 25 to 99.9 wt. %, as well as individual values or sub-ranges of the aforementioned ranges. In yet other instances, the active agent is present in a concentration of greater than about 1, 5, 15, 20, 25, 30, 35, 40, 45, or 50 wt. % of the initial coating. For the aforementioned, the concentrations are after total or near total evaporation of any volatile cosmetically acceptable carrier(s) present in the nail composition and prior to undergoing any reactions, such as polymerization, of the active agent(s) in the nail composition.

In some instances, the active agent includes a functional group. Suitable functional groups include carboxylic acid, ether, ester, carboxylate, amide, amine, alkyl, alkenyl, alkynyl, aryl, hydroxyl, anhydride, acrylate, methacrylate, acrylamide, methacrylamide, maleimide, epoxide, imide, lactone, urea, siloxy, thioether, disulfide, polysulfide, ketone, aldehyde, and combinations thereof.

In some instances, the at least one cyclic ring containing a disulfide or polysulfide bond is a dithiolane, such as a 1,2-dithiolane, asparagusic acid, methyl asparagusic acid, α-lipoic acid, esters of α-lipoic acid (such as methyl lipoate and ethyl lipoate), amides of α-lipoic acid, salts of α-lipoic acid, or combinations thereof. In some other instances, the at least one cyclic ring containing a disulfide or polysulfide bond is a dithiane, such as a 1-2,dithiane, 4,5-dihydroxy-1,2-dithiane, ethers or esters of 4,5-dihydroxy-1,2-dithiane, or combinations thereof. In still other instances, the at least one cyclic ring containing a disulfide or polysulfide bond is a dithiepane, such as 1-2,dithiepane, 1-oxa-4,5-dithiacycloheptane, 1,2-dithiapan-4-ol, or combinations thereof. In some other instances, the active agent is or includes a poly(disulfide), where such poly(disulfide)s can be synthesized via either step-growth polymerization, such as polycondensation of dihalides with sulfur, or oxidative polymerization of dithiols. Examples include, without limitation, the polymer formed by oxidation of 2-[2-(2-sulfanylethoxy)ethoxy]ethanethiol (DODT) and other systems discussed in “Green Polymer Chemistry: Living Dithiol Polymerization via Cyclic Intermediates”, by Emily Q. Rosenthal, Judit. E. Puskas, and Chrys Wesdemiotis, Biomacromolecules 2012, 13, 1, 154-164. In some instances, the active agent can undergo at least partial auto-polymerization, for example, via a cationic mechanism, to form poly(disulfide)-containing oligomers or polymers.

In some particular instances, the active agent is α-lipoic acid, an ester of α-lipoic acid, an anhydride of α-lipoic acid, a salt of α-lipoic acid, or an amide of α-lipoic acid.

In some instances, the active agent is 1,2-dithiolane (such as lipoic acid), asparagusic acid, 1,2,3-trithiolane, S₈, 1,2-dithiane, 1,2-dithepane, or combinations thereof.

In certain instances, the molecular structure of the active agent is according to Formula I:

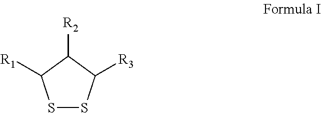

where at least one of R₁, R₂, and R₃ groups is a moiety independently selected from substituted or unsubstituted alkyl, substituted or unsubstituted alkenyl, substituted or unsubstituted alkynyl, substituted or unsubstituted aryl, carboxylic acid (—COOH), —C(O)R^{a}, —C(O)OR^{a}, carboxylate (—COO⁻), primary amide (e.g., —C(O)NH₂), secondary amide (e.g., —C(O)NHR^{a}), —C(O)NR^{a}R^{b}, —NR^{a}R^{b}, hydroxyl (—OH), anhydride (—C(O)—O—C(O)R^{a}), acrylate, methacrylate, acrylamide, styrene, substituted styrene, maleimide, epoxide, imide, lactone, urea (—NR^{a}—C(O)—NR^{a}R^{b}), ether (—OR^{c}), siloxy, thioether (—SR^{c}), ketone, thioketone, selenoketone, and combinations thereof; wherein R₁, R₂, and R₃ are hydrogen when they are not one of the aforementioned moieties;
wherein R^{a} and R^{b} are each independently selected from hydrogen, alkyl, alkenyl, alkynyl, aryl, cycloalkyl, aryl, heterocycloalkyl and heteroaryl; wherein each of R^{a} and R^{b} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, siloxy, alkoxycarbonyl, aminocarbonyl, alkylaminocarbonyl and dialkylaminocarbonyl; wherein R^{c} is selected from alkyl, alkenyl, alkynyl, aryl, cycloalkyl, aryl, heterocycloalkyl and heteroaryl; wherein R^{c} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

In some instances, for active agents of Formula I the alkyl, alkenyl, alkynyl, or aryl moieties each independently include one or more substituents selected from carboxylic acid (—COOH), ester, amide, carboxylate (—COO⁻), halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof. In some instances, for active agents of Formula I, R₁ and R₃ are hydrogens and R₂ is a linear C₄ alkyl that is optionally substituted, such as with a terminal carboxylic acid (COOH) group, such as forming lipoic acid. In some instances, for active agents of Formula I, R₁ is —C(O)OR^{a} and R^{a} is selected from hydrogen, alkyl, alkenyl, alkynyl, aryl, cycloalkyl, aryl, heterocycloalkyl and heteroaryl; where the R^{a} group is optionally substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, siloxy, alkoxycarbonyl, aminocarbonyl, alkylaminocarbonyl and dialkylaminocarbonyl.

In other instances, the molecular structure of the active agent is according to Formula II:

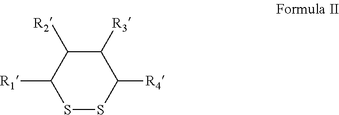

where at least one of R₁′, R₂′, R₃′, and R₄′ is a moiety independently selected from substituted or unsubstituted alkyl, substituted or unsubstituted alkenyl, substituted or unsubstituted alkynyl, substituted or unsubstituted aryl, carboxylic acid (—COOH), —C(O)R^{a}, —C(O)OR^{a}, carboxylate (—COO⁻), primary amide (e.g., —C(O)NH₂), secondary amide (e.g., —C(O)NHR^{a}), —C(O)NR^{a}R^{b}, —NR^{a}R^{b}, hydroxyl (—OH), anhydride (—C(O)—O—C(O)R^{a}), acrylate, methacrylate, acrylamide, styrene, substituted styrene, maleimide, epoxide, imide, lactone, urea (—NR^{a}—C(O)—NR^{a}R^{b}), ether (—OR^{c}), siloxy, thioether (—SR^{c}), ketone, thioketone, selenoketone, and combinations thereof; wherein the R₁′, R₂′, R₃′, and R₄′ groups are hydrogen when they are not one of the aforementioned moieties; where R^{a} and R^{b} are each independently selected from hydrogen, alkyl, alkenyl, alkynyl, aryl, cycloalkyl, aryl, heterocycloalkyl and heteroaryl; wherein each of R^{a} and R^{b} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, siloxy, alkoxycarbonyl, aminocarbonyl, alkylaminocarbonyl and dialkylaminocarbonyl; where R^{c} is selected from alkyl, alkenyl, alkynyl, aryl, cycloalkyl, aryl, heterocycloalkyl and heteroaryl; wherein R^{c} is optionally independently substituted with one or more substituents selected from halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof. In some instances, a double bond or substituted double bond may be present between R₂′ and R₃′, such as 3,6-dihydro-1,2-dithiine, and where R₂′ and R₃′ may be hydrogen or another group from those listed above.

In some instances, for active agents of Formula II the alkyl, alkenyl, alkynyl moieties each independently include one or more substituents selected from the group consisting of carboxylic acid (—COOH), ester, amide, carboxylate (—COO⁻), halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

In still other instances, the molecular structure of the active agent is according to Formula III:

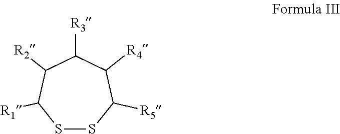

where at least one of R₁″, R₂″, R₃″, R₄″, and R₅″ is a moiety independently selected from substituted or unsubstituted alkyl, substituted or unsubstituted alkenyl, substituted or unsubstituted alkynyl, substituted or unsubstituted aryl, carboxylic acid (—COOH), —C(O)R^{a}, —C(O)OR^{a}, carboxylate (—COO⁻), primary amide (e.g., —C(O)NH₂), secondary amide (e.g., —C(O)NHR^{a}), —C(O)NR^{a}R^{b}, —NR^{a}R^{b}, hydroxyl (—OH), anhydride (—C(O)—O—C(O)R^{a}), acrylate, methacrylate, acrylamide, styrene, substituted styrene, maleimide, epoxide, imide, lactone, urea (—NR^{a}—C(O)—NR^{a}R^{b}), ether (—OR^{c}), siloxy, thioether (—SR^{c}), ketone, thioketone, selenoketone, and combinations thereof; wherein the R₁″, R₂″, R₃″, R₄″, and R₅″ groups are hydrogen when they are not one of the aforementioned moieties; where R^{a} and R^{b} are each independently selected from hydrogen, alkyl, alkenyl, alkynyl, aryl, cycloalkyl, aryl, heterocycloalkyl and heteroaryl; wherein each of R^{a} and R^{b} is optionally independently substituted with one or more substituents selected from halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, siloxy, alkoxycarbonyl, aminocarbonyl, alkylaminocarbonyl and dialkylaminocarbonyl; where R^{c} is selected from alkyl, alkenyl, alkynyl, aryl, cycloalkyl, aryl, heterocycloalkyl and heteroaryl; wherein R^{c} is optionally independently substituted with one or more substituents selected from halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

In some instances, for active agents of Formula III the alkyl, alkenyl, alkynyl moieties each independently include one or more substituents selected from carboxylic acid (—COOH), ester, amide, carboxylate (—COO⁻), halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

In yet other instances, the molecular structure of the active agent is according to Formula IV:

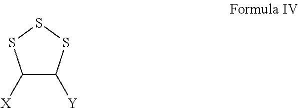

where at least one of X and Y is a moiety independently selected from substituted or unsubstituted alkyl, substituted or unsubstituted alkenyl, substituted or unsubstituted alkynyl, substituted or unsubstituted aryl, carboxylic acid (—COOH), —C(O)R^{a}, —C(O)OR^{a}, carboxylate (—COO⁻), primary amide (e.g., —C(O)NH₂), secondary amide (e.g., —C(O)NHR^{a}), —C(O)NR^{a}R^{b}, —NR^{a}R^{b}, hydroxyl (—OH), anhydride (—C(O)—O—C(O)R^{a}), acrylate, methacrylate, acrylamide, styrene, substituted styrene, maleimide, epoxide, imide, lactone, urea (—NR^{a}—C(O)—NR^{a}R^{b}), ether (—OR^{c}), siloxy, thioether (—SR^{c}), ketone, thioketone, selenoketone, and combinations thereof; wherein the X and Y groups are hydrogen when they are not one of the aforementioned moieties; where R^{a} and R^{b} are each independently selected from hydrogen, alkyl, alkenyl, alkynyl, aryl, cycloalkyl, aryl, heterocycloalkyl and heteroaryl; wherein each of R^{a} and R^{b} is optionally independently substituted with one or more substituents selected from halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, siloxy, alkoxycarbonyl, aminocarbonyl, alkylaminocarbonyl and dialkylaminocarbonyl; where R^{c} is selected from alkyl, alkenyl, alkynyl, aryl, cycloalkyl, aryl, heterocycloalkyl and heteroaryl; where R^{c} is optionally independently substituted with one or more substituents selected from halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, haloalkyl, hydroxyalkyl, carboxy, alkoxy, aryloxy, siloxy, alkoxycarbonyl, aminocarbonyl, alkylaminocarbonyl, dialkylaminocarbonyl, and combinations thereof.

In some instances, for active agents of Formula IV the alkyl, alkenyl, alkynyl moieties each independently comprise one or more substituents selected from carboxylic acid (—COOH), ester, amide, carboxylate (—COO⁻), halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

In some instances, the active agent is a multifunctional compound which includes one or more moieties, wherein each of the one or more moieties includes at least one cyclic ring containing a disulfide or polysulfide bond. In some instances, the one or more moieties are or include structures defined by Formulas I-IV above. For example, a tris(dithiolane) can be formed by esterifying glycerin with lipoic acid. Such multi-functional compounds can be fully bio-based, for example, by esterifying sugars or polysaccharides with lipoic acid. In other examples, the multi-functional compounds are synthesized by the esterification of lipoic acid with pentaerythritol, dipentaerythritol, alpha-cyclodextrin, beta-cyclodextrin, gamma-cyclodextrin, ethylene glycol, poly(ethylene glycol), poly(ethylene oxide), poly(vinyl alcohol), poly(tetrahydrofuran), or polyesters such as polylactide and poly(caprolactone), and the like. In other examples, the multi-functional compounds are synthesized by the amidation of lipoic acid with ethylenediamine, tris(2-aminoethyl)amine, diethylenetriamine, triaminononane, Jeffamines, poly(ethyleneimine), and the like.

In some instances, the active agent(s) are attached to a polymer backbone. Exemplary suitable polymer backbones include but are not limited to polysiloxane, polyester, polyether, polyacrylate, polymethacrylate, polyacrylamide, polymethacrylamide, polyethylene, polypropylene, poly(vinyl alcohol), poly(vinyl acetate), polystyrene, polyolefin, polyurethane, polycarbonate, polyamide, polyurea, polypeptide, polypeptoid, and polyamine polymer backbones, as well as copolymers thereof.

The nail compositions may include monomeric active agents, polymers formed therefrom, or combinations thereof.

In some instances, the nail compositions include an active agent that is a monomer capable of being polymerized.

In certain other instances, the nail compositions include an active agent that has been at least partially or fully polymerized, where the active agent includes disulfide or polysulfide bonds without the need for exposure to an external stimulus. Such nail compositions include the active agent in a polymerized form (i.e., partially polymerized, fully polymerized, co-polymerized, or combinations thereof). In such instances, after the coating is deposited on the nail, it may or may not require further curing via a suitable stimulus, such as heat or light. In such instances, the pre-polymerized polymer can be made from the active agents described above and can, in some instances, also be co-polymerized with other reactive co-monomers, as described further below.

In certain other instances, the nail compositions include an active agent that has been at least partially or fully polymerized, and optionally an active agent that is a monomer capable of being polymerized. In some instances, the at least partially or fully polymerized polymer resulting from the active agent can be selected, without limitation, from poly(lipoic acid), poly(alkyl lipoate), poly(lipoamide), a poly(disulfide) formed by condensation polymerization, a poly(disulfide) formed by oxidative coupling, a poly(disulfide) formed by ring opening polymerization, a poly(disulfide) formed by autopolymerization, a poly(disulfide) formed by cationic polymerization, a poly(disulfide) formed by anionic polymerization, a poly(disulfide) formed by radical polymerization, or copolymers thereof. Examples of thiols used for the polymer synthesis include, but are not limited to, dithiols, trithiols, tetrathiols, and polythiols, such as: 1,1-propanedithiol, 1,2-propanedithiol, 1,3-propanedithiol, 1,4-butanedithiol, 1,5-pentanedithiol, 1,6-hexanedithiol, benzene-1,2-dithiol, benzene-1,3-dithiol, benzene-1,4-dithiol, 4,4′-thiobisbenzenethiol, 1,2-pentanedithiol, ethanedithiol, pentaerythritol tetrakis(3-mercaptopropionate), biphenyl-4,4′-dithiol, 1,4-bis(4-mercaptophenyl)benzene, 1,3,4-thiadiazole-2,5-dithiol, (1,2,4) thiadiazole-3,5-dithiol, poly(ethylene glycol) dithiol, 1,3,5-trimercaptobenzene, pentaerythritol tetra(2-mercaptoacetate), 4-mercaptomethy-1,8-dimercapt-3,6-dithiaoctane, 5,7-dimercaptomethyl-1,11-dimercapto-3,6-trithiaundecane, bis(mercaptoethyl)sulfide, 2,5-bis(mercaptomethyl)-1,4-dithiane, tetra(ethylene glycol) dithiol, 2,2′-(ethylenedioxy)diethanethiol, 1,1′,4′,1″-terphenyl-4-thiol, 2-thiazoline-2-thiol, 5-bromopyridine-2-thiol, biphenyl-4-thiol, 1,3,5-benzenetrithiol, 1,7-naphthalenedithio, 1,5-naphtalenedithiol, and the like. Examples of cyclic disulfides which can be used for polymer synthesis include, but are not limited to, lipoic acid, ethyl lipoate, 1,2-dithiane, asparagusic acid, 1,4,5-oxadithiepan-2-one, and the like.

For the active agents, such as of Formulae I-IV, a single substituent (i.e., R, X, Y) is shown at each carbon of the heterocycle. However, it is understood that one or both positions at each carbon may be substituted by the respective R groups shown at the positions of Formulae I-IV where the R groups are independently selected from the recited groups listed above. For example, an active agent of Formula I above may include additional R groups in lieu of the hydrogens at each position such that there are up to six R groups in an agent of Formula I and each R group is independently selected from the recited groups listed above. Further, the skilled person further understands that structures of Formulae I-IV may include one or more chiral centers and thus exist as one or more stereoisomers. As used herein, the term “stereoisomers” refers to compounds made up of the same atoms having the same bond order but having different three-dimensional arrangements of atoms which are not interchangeable. The term “chiral center” refers to a carbon atom to which four different groups are attached.

Compounds of Formulae I-IV, multifunctional compounds, and other active agents described herein can be obtained from commercial sources or can be produced by the person of ordinary skill in the art of synthetic chemistry.

In some instances, the nail composition comprises a plurality of the active agents described herein.

In some instances, the nail compositions are anhydrous compositions. The term “anhydrous composition” refers to a composition comprising less than 5% by weight of water, or less than 2% by weight of water, or less than 1% by weight of water relative to the total weight of the composition. In some instances, an anhydrous composition is free of water. For example, the nail composition may be an anhydrous composition, optionally where the nail composition is in the form of a powder.

In some instances, the nail compositions are fluids. Such fluid nail compositions can have a viscosity measured at 25° C. and at atmospheric pressure which is less than or equal to 4.5 Pa·s, between 1 mPa·s and 4.5 Pa·s at a shear rate of 200 s- and less than or equal to 50 Pa·s using a Brookfield Rheomat RM 180 viscometer equipped with a No. 4 spindle, the measurement being taken after 10 minutes of rotation of the spindle in order to stabilize the rotational speed and the viscosity. Other viscosities for the nail composition are possible such as within the range 0.5 cP to 25.000 cP. In some instances, the nail compositions for application to the nail surface are: (1) low viscosity nail compositions having viscosities in a range of about 300 to 800 cP: (2) medium viscosity nail compositions having viscosities ranging from between about 800 to 1500 cP; or (3) high viscosity nail compositions having viscosities in a range from about 1500 to 1000 cP 1500 to 10000 cP, or 1500 to 100000 cP. Individual values or sub-ranges of any of the aforementioned viscosity ranges are possible.

### A. Optional Co-Reactive Monomers

As noted above, the active agents of the nail compositions are capable of undergoing a polymerization reaction when exposed to a stimulus, such as light or heat, to form a nail coating on a nail surface. In some instances, the nail compositions may further include one or more co-reactive monomers capable of undergoing copolymerization or condensation with the active agent. Such co-reactive monomers can be selected, without limitation, from acrylates, multifunctional acrylates (such as polyethylene glycol (200) diacrylate, polypropylene glycol (400) diacrylate, polytetramethylene glycol (650) diacrylate, tricyclodecane dimethanol diacrylate, ethoxylated bisphenol A diacrylate, ethoxylated trimethylolpropane triacrylate, ethoxylated glycerin triacrylate, pentaerythritol tri- or tetraacrylate, di(trimethylolpropane) tetraacrylate, dipentaerythritol penta-/hexa-acrylate, polypentaerythritol polyacrylate), diacrylates, triacrylates, tetraacrylates, pentaacrylates, hexaacrylates, bis and tris(2-acryloxyethyl)isocyanurate, acrylamides, methacrylates, isocyanates, methacrylates, vinyl acetates, vinyl ethers (e.g., methyl vinyl ether, ethyl vinyl ether, propyl vinyl ether, butyl vinyl ether, and isobutyl vinyl ether), multi-functional vinyl ethers such as diethylene glycol divinyl ether, mono-alkenes, bis-alkenes, tris-alkenes, tetrakis-alkenes, pentakis-alkenes, polyurethane acrylates (such as supplied by Bomar), polyester acrylates (such as supplied by Bomar), polyether acrylates (such as supplied by Bomar), norbornene, substituted norbornenes, dicyclopentadiene, carboxylic acids, poly(carboxylic acids), epoxides, poly(epoxides), alcohols, polyols, amines, polyamines, multifunctional thiols, and combinations thereof. In some instances, the one or more co-reactive monomers include more than 5 alkene groups, lactones, epoxides, styrenics (i.e., a compound, polymer, or material that is derived from styrene, such as divinylstyrenics), and/or lactides thereon. In some instances, the one or more co-reactive monomers are selected from a linear or branched alkyl acrylate, methyl acrylate, ethyl acrylate, propyl acrylate, butyl acrylate, 2-ethylhexyl acrylate, isobornyl acrylate, 2-hydroxyethyl acrylate, hydroxypropyl acrylate, a linear or branched alkyl methacrylate, methyl methacrylate, ethyl methacrylate, propyl methacrylate, butyl methacrylate, isobornyl methacrylate, 2-ethylhexyl methacrylate, hydroxypropyl methacrylate, 2-hydroxyethyl methacrylate, styrene, and combinations thereof. In some instances, the co-reactive monomers are selected from n-butyl acrylate, n-butyl methacrylate, 2-hydroxyethyl acrylate, 2-hydroxyethyl methacrylate, trimethylolpropane triacrylate, pentaerthyritol tetrakis(3-mercaptopropionate), dipentaerythritol pentahexa-acrylate, pentaerythritol tetraacrylate, tripropylene glycol diacrylate, and combinations thereof.

In some instances, the one or more co-reactive monomers are present in a concentration ranging from about 0.1 to 99 wt. % or 1 to 99 wt. % of the total weight of the nail composition, as well as individual concentration values or sub-ranges contained within. In some instances, the one or more co-reactive monomers are present in a concentration ranging from about 0.1 to 10 wt. %, 0.1 to 5 wt. %, 0.1 to 2.5 wt. %, 0.1 to 1 wt. %, or 0.3 to 3 wt. % of the total weight of the nail composition, as well as individual concentration values or sub-ranges contained within.

In some instances, where the one or more co-reactive monomers are present the active agent and the co-reactive monomers are present in the nail composition at weight ratios of active agent(s) to co-reactive monomers in the range of 1:10 to 100:1. In some instances, the one or more co-reactive monomers are present in the nail composition at weight ratios of active agent(s) to co-reactive monomers in the range of 40:1 to 8:1. Individual ratios and sub-ranges of the aforementioned ratio ranges are possible.

In some instances, in the initial coating formed following application to a nail surface and evaporation of the volatile cosmetically acceptable carrier(s) has a weight ratio of active agent(s) to co-reactive monomers in the range of 40:1 to 8:1. Individual ratios and sub-ranges of the aforementioned ratio range are possible. In some instances, the amount of active agent in the initial coating is greater than about 50% than the nail composition.

In some other instances, the nail compositions may further include one or more (second) monomers capable of undergoing an independent polymerization or condensation and do not co-polymerize with the active agent monomer. In other words, the one or more co-reactive monomers can form an independent polymer which does not include any repeat units of the active agent and at least two types of polymers are formed in the nail coating. Zero, one, or both of these distinct types of polymers could form independent networks. Suitable and non-limiting co-reactive monomers, in such instances, can be selected from methacrylates, cyanoacrylates, acrylates or other vinyl derivatives, or pairs of multifunctional monomers that undergo step-growth polymerization, such as amines and isocyanates, alcohols and isocyanates, azides and alkynes, nucleophiles and sulfonyl fluorides, carboxylic acids and alcohols, activated carboxylic acids and alcohols, esters and alcohols, acid chlorides and amines, or acid chlorides and alcohols, and amines and carboxylic acids. Non-limiting exemplary polymer(s) can include polyacrylates, polymethacrylates, polyurethanes, polyamides, polycyanoacrylates, polyacrylamides, polyesters, epoxies, siloxanes, and/or styrenics.

The nail compositions described above may take any suitable form. In some instances, the nail composition is in the form of a nail strengthener, a nail primer, or a nail protector. In some instances, the nail composition is in the form of a nail polish, a nail lacquer, a nail gel, nail extension, or a dip nail. In some instances, the nail composition is in a form suitable for use as a base coat, a color coat, or a top coat. In some instances, the nail compositions can be used to form gel nails. In other instances, the nail compositions can be used in a dipping powder process. Gel nail and dip powder processes are known to those skilled in the art and the nail compositions described may take the form of the various coats or powder used in such processes.

In some instances, the nail compositions are provided and/or stored in a container which reduces or blocks exposure of light (such as ultraviolet light) to the nail formulation contained within.

### B. Cosmetically Acceptable Excipients

Common nail composition excipients are described in “Polish College: The Basics—An introduction to common nail polish ingredients.” by Doug Schoon, Dr. Vivian, B. Valenty, and Paul Brys Jun. 1, 2008) which are incorporated in relevant part herein. Without limitation, some exemplary excipients can include: humectants, emollients, oils, moisturizers, vitamins, and/or fragrances. Other excipients can include: plasticizers, which can make the compositions more flexible and increase the durability of the nail coating; non-limiting plasticizers can be dibutyl phthalate (DBP) and camphor; solvents and carriers, which can help make the liquid nail compositions spreadable and can keep the ingredients dissolved in the composition during application, but evaporate away after the composition has been applied. Solvents evaporate at different rates, so combinations of solvents can be used together to create an ideal evaporation time. Solvents include, without limitation, acetone, ethyl acetate, ethanol, isopropanol, propyl acetate, butyl acetate, amyl acetate, isoamyl acetate, toluene, and combinations thereof. The nail compositions may also contain water in any suitable amount and in combination with other solvents or carriers.

In some particular instances, the nail composition further includes one or more cosmetically acceptable excipients selected from humectants, emollients, oils, moisturizers, and/or fragrances.

In some instances, the one or more cosmetically acceptable excipients may be selected from a vitamin (such as vitamin E), an oil (such as jojoba oil and/or white eucalyptus oil), and combinations thereof.

In some instances, the one or more cosmetically acceptable excipients are present in a concentration ranging from about 0.001 to 99 wt. % of the total weight of the nail composition, as well as individual concentration values or sub-ranges contained within.

i. Pigments/Dyes for Nail Compositions

The nail compositions may include one or more pigments or dyes to provide a color to the nail coating formed. In some instances, the nail compositions may exclude such pigments or dyes. Optionally, when dyes or pigments are present in the nail composition, they do not provide a color to the final coating formed but merely color the composition for sale. Combinations of naturally occurring and manufactured pigments/dyes can be blended together to create a desired shade or hue.

In some instances, the pigments or dyes may be chosen from coated or uncoated pigments, water-soluble dyes, liposoluble dyes, and mixtures thereof. The term “pigments” means white or colored, mineral or organic particles, which are insoluble in an aqueous medium, and which are intended to color and/or opacify the nail composition and/or a coating formed thereof.

Suitable mineral pigments that may be used in the compositions include, but are not limited to, zirconium oxide or cerium oxide, zinc oxide, iron oxide (black, yellow or red) or chromium oxide, manganese violet, ultramarine blue, chromium hydrate and ferric blue, titanium dioxide, and metal powders, for instance aluminum powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS. In some instances, the size of the pigment that is useful may generally be greater than about 10 nm, greater than 25 nm, greater than 50 nm, or greater than 100 nm and may range from about 10 nm to 10 microns, 50 to 10 microns, 100 to 10 microns, 200 nm to 5 microns, or 300 nm to 1 micron. In some instances, the pigments have a size characterized by a D[50] greater than 100 nm and possibly ranging up to 10 microns, from 200 nm to 5 microns, or from 300 nm to 1 micron; where D[50] represents the maximum size that 50% by volume of the particles have. The sizes are measured by static light scattering using a commercial MasterSizer 3000® particle size analyzer from Malvern, which makes it possible to determine the particle size distribution of all of the particles over a wide range which may extend from 10 nm to 1000 microns. The data are processed on the basis of the standard Mie scattering theory, which is suitable for size distributions ranging from submicron to multimicron and allows an “effective” particle diameter to be determined. See, e.g., Van de Hulst, H. C., Light Scattering by Small Particles. Chapters 9 and 10, Wiley, New York, 1957. Individual size values or sub-ranges of the aforementioned ranges are possible.

In some instances, the nail compositions also include nacres. The term “nacres” is understood as meaning colored particles of any form, which may or may not be iridescent, notably produced by certain mollusks in their shell, or alternatively synthesized, and which have a color effect via optical interference. The nacres may be chosen from nacreous pigments, such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic dyes. Without limitation, examples of nacres that may also be mentioned include natural mica covered with titanium oxide, with iron oxide, with natural pigment or with bismuth oxychloride. The nacres may have a yellow, pink, red, bronze, orange, brown, gold and/or coppery color or tint.

In some instances, the nail compositions include one or more organic pigments or dyes. The term “organic pigment” refers to any pigment that satisfies the definition in Ullmann's encyclopedia in the chapter on organic pigments. The organic pigment be chosen, without limitation, from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, and quinophthalone compounds. In certain instances, the organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100, and 74160, the yellow pigments codified in the Color Index under the references CI 11680, 11710, 15985, 19140, 20040, 21100, 21108, 47000, and 47005, the green pigments codified in the Color Index under the references CI 61565, 61570, and 74260, the orange pigments codified in the Color Index under the references CI 11725, 15510, 45370, and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, and 75470, and the pigments obtained by oxidative polymerization of indolic or phenolic derivatives. Suitable organic dyes, include, without limit, cochineal carmine, D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), and D&C Blue 1 (CI 42 090).

Suitable pigments may also be in the form of composite pigments which are composed of particles including a mineral core at least partially covered with an organic pigment and at least one binder for fixing the organic pigments to the core.

In some other instances, the pigment may also be a lake pigment. A lake pigment is a pigment made by precipitating a dye with an inert binder, or mordant, usually a metallic salt; i.e., an insolubilized dye adsorbed onto insoluble substrates/particles, the assembly thus obtained remaining insoluble during use. The inorganic substrates onto which the dyes are adsorbed can be, for example, alumina, silica, calcium sodium borosilicate or calcium aluminum borosilicate and aluminum.

As noted above, the dyes can be water-soluble dyes. Water soluble dyes refer to any natural or synthetic, generally organic compound, which is soluble in an aqueous phase or water-miscible solvents and which is capable of imparting color. Non-limiting examples of water-soluble dyes can include, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, and FDC Blue 1. Suitable natural water-soluble dyes include anthocyanins.

As noted above, the dyes can also be liposoluble dyes, which refer to any natural or synthetic, generally organic compound, which is soluble in an oily phase or in solvents that are miscible with the oily phase, and which is capable of imparting color. Non-limiting examples of liposoluble dyes can include, for instance, DC Red 17, DC Red 21, DC Red 27, DC Green 6, DC Yellow 11, DC Violet 2, DC Orange 5, Sudan red, and Sudan brown. Other natural liposoluble dyes, can include, without limit, carotenes, for instance 3-carotene, a-carotene and lycopene; quinoline yellow; xanthophylls such as astaxanthin, antheraxanthin, citranaxanthin, cryptoxanthin, canthaxanthin, diatomoxanthin, flavoxanthin, fucoxanthin, lutein, rhodoxanthin, rubixanthin, siphonaxanthin, violaxanthin, zeaxanthin; annatto; curcumin; quinizarin (ceres green BB, D&C green No. 6, 01 61565, 1,4-di-p-toluidinoanthraquinone, green No. 202, quinazine green SS) and chlorophylls.

In some instances, the pigments or dyes which may be part of the nail compositions described prior to application to a nail surface may be present at concentrations of less than about 50%, 40%, 30%, 20%, 10%, 5%, 4%, 3%, 2%, 1%, 0.5%, 0.1%, or 0.01% by weight relative to the total weight of nail composition. In some instances, the pigments or dyes which may be part of the nail compositions described prior to application to a nail surface may be present at concentrations ranging from about 0.01% to 50% by weight relative to the total weight of nail composition, as well as individual values or sub-ranges contained within. In some instances, the pigments or dyes that are in the nail coating formed following application and, optional curing on a nail surface are present at concentrations of less than about 50%, 40%, 30%, 20%, 10%, 5%, 4%, 3%, 2%, 1%, 0.5%, 0.1%, or 0.01% by weight relative to the total weight of nail coating. In some instances, the pigments or dyes in the nail composition (i.e., prior to application to a nail surface) are present at concentrations ranging from about 0.01% to 50% by weight relative to the total weight of nail coating, as well as individual values or sub-ranges contained within.

ii. Solvents, Carriers, and Diluents

Various solvents, carriers, and/or diluents, which are typically liquids, such as water (deionized, distilled or purified), alcohols, fatty alcohols, polyols, low molecular weight polymers (polymers with weight average molecular weights below about 10,000 g/mol such as dimethicone, poly(ethylene glycol), high molecular weight polymers (polymers with weight average molecular weights above about 10,000 g/mol, such as poly(methyl methacrylate), acrylate copolymers, and the like), and mixtures thereof can be used in the nail compositions. The solvent and/or carrier are selected to evaporate when the nail composition is applied to the nail surface. The diluent can be non-volatile and remain in the nail coating after application. In some instances, non-aqueous or hydrophobic auxiliary solvents can be used to prepare compositions that are substantially water-free products, such as nail lacquers. Non-limiting examples of solvents and carriers, other than water, can include linear, cyclic, and branched alcohols, such as ethanol, propanol, isopropanol, hexanol, cyclohexanol and the like; aromatic alcohols, such as benzyl alcohol and the like; saturated C₁₂ to C₃₀ fatty alcohols, such as lauryl alcohol, myristyl alcohol, cetyl alcohol, stearyl alcohol, behenyl alcohol, and the like. Non-limiting examples of polyols include polyhydroxy alcohols, such as glycerin, propylene glycol, butylene glycol, hexylene glycol. C₂ to C₄ alkoxylated alcohols and C₂ to C₄ alkoxylated polyols, such as ethoxylated, propoxylated, and butoxylated ethers of alcohols, diols, and polyols having about 2 to about 30 carbon atoms and 1 to about 40 alkoxy units, polypropylene glycol, polybutylene glycol, and the like. Non-limiting examples of non-aqueous diluents include silicones, and silicone derivatives, such as dimethicone, and the like; natural and synthetic oils and waxes, such as vegetable oils, plant oils, animal oils, essential oils, mineral oils, C₁₅ to C₄₀ isoparaffins, alkyl carboxylic esters, such as glucose monoterpene esters, jojoba oil, shark liver oil, and the like. Combinations of various solvents, carriers, and/or diluents are possible.

In some instances, the solvents, carriers, and/or diluents in the nail compositions are at a total concentration of at least 0.5% and less than about 99%, 90%, 80%, 70%, 60%, 50%, 40%, 30%, 20%, 10%, 5%, or 1% by weight relative to the total weight of nail composition. In some instances, the solvent, carriers, and/or diluents in the nail compositions are present at a total concentration ranging from about 1% to 99% by weight relative to the total weight of nail composition, as well as individual values or sub-ranges contained within. Total concentration refers to the concentration of all of the solvents, carriers, and diluents that are present in the nail composition combined.

In some particular instances, the cosmetically acceptable carrier of the nail compositions is selected to be a volatile solvent. Without limitation, exemplary volatile solvents can be selected from acetone, ethyl acetate, propyl acetate, butyl acetate, amyl acetate, isobutyl acetate, isoamyl acetate, ethanol, isopropanol, water, and combinations thereof. In other instances, the volatile solvent is selected from the group consisting of acetone, methyl acetate, ethyl acetate, propyl acetate, butyl acetate, isopropyl acetate, isobutyl acetate, pentyl acetate, isopentyl acetate, hexyl acetate, benzyl acetate, heptyl acetate, octyl acetate, nonyl acetate, methyl ethanoate, ethyl ethanoate, propyl ethanoate, butyl ethanoate, isopropyl ethanoate, isobutyl ethanoate, pentyl ethanoate, isopentyl ethanoate, hexyl ethanoate, benzyl ethanoate, heptyl ethanoate, octyl ethanoate, nonyl ethanoate, methyl propanoate, ethyl propanoate, propyl propanoate, butyl propanoate, isopropyl propanoate, isobutyl propanoate, pentyl propanoate, isopentyl propanoate, hexyl propanoate, benzyl propanoate, heptyl propanoate, octyl propanoate, nonyl propanoate, ethanol, isopropanol, water, and combinations thereof. In some instances, the volatile solvent is acetone. In some other instances, the volatile solvent is an ester compound, such as those named above.

In some instances, the cosmetically acceptable carrier of the nail composition is in a concentration ranging from about 1 wt. % to 99 wt. % of the total weight of the nail composition, as well as individual values or sub-ranges contained within.

In some instances, initial coating can be formed prior to any reaction of the active agent(s) and optional co-reactive monomers present in the initial coating. Typically, a nail coating forms due to a reaction, such as a polymerization reaction, which forms the nail coating, which can be different than the initial coating. In some instances, the initial coating deposited by an already polymerized or partially polymerized active agent is considered a nail coating. In some instances, the nail coating formation process (by reaction of the active agent(s) and optional co-reactive monomers) may occur during or following the evaporation of any volatile carrier(s) present in the nail composition. In some instances, the active agent is present in a concentration of greater than about 50 wt. % of the initial coating resulting from evaporation of all or substantially all of the volatile solvent of the nail composition, such as prior to exposure to any stimulus.

iii. Optional Photochemical and/or Thermal Initiators

In some instances, the nail compositions described herein further include one or more photochemical and/or thermal initiators. In some instances, the one or more photochemical and/or thermal initiators are radical initiators selected, without limitation, from type-1 or type-2 photoinitiators, such as benzoin ethers, benzil ketals, α-dialkoxy-acetophenones, α-hydroxy-alkylphenones, α-aminoalkyl-phenones, acyl phosphine oxides, benzophenones, thioxanthones, α-diketones such as camphorquinone and derivatives thereof, α-hydroxyketones, and/or titanocenes. In some instances, coinitiators such as amines can be used to improve the reactivity of photoinitiators like camphorquinone or derivatives thereof. In other instances, the one or more photochemical and/or thermal initiators are selected, without limitation, from 2,2-dimethoxy-2-phenylacetophenone; phosphine oxides and derivatives including phosphinates, such as phenylbis(2,4,6-trimethylbenzoyl)phosphine oxide, diphenyl(2,4,6-trimethylbenzoyl)phosphine oxide, ethyl (2,4,6-trimethylbenzoyl)phenylphosphinate, lithium phenyl-2,4,6-trimethylbenzoylphosphinate, and bis(2,6-dimethoxybenzoyl)(2,4,4-trimethylpentyl) phosphine oxide; 2-hydroxy-2-methylpropiophenone, camphorquinone, coumarin, 1-hydroxycyclohexyl phenyl ketone, peroxides (such as benzoylperoxide), azo initiators (such as azobisisobutyronitrile), benzophenone, ring-opening polymerization initiators (such as thiolates), ring opening polymerization sulfonium-ion initiators, photoacid generators, photobase generators, and combinations thereof. The one or more photochemical and/or thermal initiators may be present in a concentration ranging from about 0.0001 wt. % to 20 wt. % of the total weight of the nail composition, as well as individual values or sub-ranges contained within.

iv. Optional Stabilizing Agent(s), Thiol-Capping Agent(s), and/or UV Absorbing Agent(s)

In some instances, the nail compositions further include at least one stabilizing agent. The stabilizing agent can reduce or prevent changes in odor, appearance, composition and/or viscosity over time compared to the same composition in the absence of the stabilizing agent(s) over the same period of time, such as for a period of time ranging from at least about 3 to 24 months or 6 to 12 months, as well as individual times or sub-ranges contained within the aforementioned ranges. Without limitation, stabilizing agent(s) can be hydroquinone, 2,6-di-tert-butyl-4-methylphenol (butylated hydroxytoluene, BHT), 4-tert-butylcatechol (TBC), 4-methoxyphenol (MEHQ), methylethyl ketone oxime; phosphine oxides and derivatives including phosphinates such as phenylbis(2,4,6-trimethylbenzoyl)phosphine oxide, diphenyl(2,4,6-trimethylbenzoyl)phosphine oxide, ethyl (2,4,6-trimethylbenzoyl)phenylphosphinate, lithium phenyl-2,4,6-trimethylbenzoylphosphinate, and bis(2,6-dimethoxybenzoyl)(2,4,4-trimethylpentyl) phosphine oxide; gallic acid, gallic acid esters, or a combination thereof. The stabilizing agent(s) may be present in a concentration ranging from about 0.0001 wt. % to 20 wt. % of the total weight of the nail composition, as well as individual values or sub-ranges contained within.

In some instances, the nail compositions described herein further include a thiol capping agent. The thiol capping agent can reduce or prevent changes in odor, appearance, composition and/or viscosity over time compared the same composition in the absence of the thiol capping agent(s) over the same period of time, such as for a period of at least about 3 to 24 months or 6 to 12 months, as well as individual times or sub-ranges contained within the aforementioned ranges. Without limitation, thiol capping agent(s) can be 2-iodoacetamide, 2-iodo-N-methylacetamide, 2-bromo-acetamide, methyl isocyanide, or 1,3-dimethyl-1H-pyrrole-2,5-dione, or a combination thereof. In some instances, the thiol capping agent(s) can be present in a concentration ranging from about 0.0001 wt. % to 20 wt. % of the total weight of the nail composition, as well as individual values or sub-ranges contained within.

In some instances, the nail compositions described herein further include one or more ultraviolet absorbing agents. The ultraviolet absorbing agents can absorb light having wavelengths of up to about 385 nm, up to about 415 nm, or up to about 450 nm. In some instances, the ultraviolet absorbing agents can absorb light having wavelengths in a range from about 385 to 450 nm, as well as individual values or sub-ranges contained within the aforementioned ranges. Without limitation, the one or more ultraviolet absorbing agents can be avobenzone, zinc oxide, homosalate, octocrylene, benzotriazole and derivatives thereof, “red-shifted UV absorbers” (RUVA), and 5-trifluoromethyl-2-(2-hydroxy-3-alpha-cumyl-5-tert-octylphenyl)-2H-benzotriazole (available under the trade designation “CGL-0139” from BASF, Florham Park, NJ). Other exemplary benzotriazoles can include, without limitation, 2-(2-hydroxy-3,5-di-alpha-cumylphenyl)-2H-benzotriazole, 5-chloro-2-(2-hydroxy-3-tert-butyl-5-methylphenyl)-2H-benzotriazole, 5-chloro-2-(2-hydroxy-3,5-di-tert-butylphenyl)-2H-benzotriazole, 2-(2-hydroxy-3,5-di-tert-amylphenyl)-2H-benzotriazole, 2-(2-hydroxy-3-alpha-cumyl-5-tert-octylphenyl)-2H-benzotriazole, and 2-(3-tert-butyl-2-hydroxy-5-methylphenyl)-5-chloro-2H-benzotriazole. Further exemplary RUVAs includes 2(-4,6-diphenyl-1-3,5-triazin-2-yl)-5-hexyloxy-phenol. Other exemplary UV absorbing agents can include those available from BASF under the trade designations “TINUVIN 1577,” “TINUVIN 900,” “TINUVIN 1600,” and “TINUVIN 777.” Still other exemplary UV absorbing agents are available, for example, in a polyester master batch under the trade designation “TA07-07 MB” from Sukano Polymers Corporation, Dunkin, SC. Another exemplary UV absorbing agent for poly(methyl methacrylate) is a masterbatch available, for example, under the trade designation “TA11-10 MBO1” from Sukano Polymers Corporation. Yet another exemplary UV absorbing agents for polycarbonate is a masterbatch from Sukano Polymers Corporation, under the trade designations “TA28-09 MB01.” In addition, the UV absorbers can be used in combination with hindered amine light stabilizers (HALS) and anti-oxidants. Exemplary HALS include those available from BASF, under the trade designation “CHIMASSORB 944” and “TINUVIN 123.” Exemplary anti-oxidants include those obtained under the trade designations “IRGANOX 1010” and “ULTRANOX 626”, also available from BASF. Combinations of any of the aforementioned ultraviolet absorbing agents are possible. In some instances, the one or more ultraviolet absorbing agents can be present in a concentration ranging from about 0.0001 wt. % to 20 wt. % of the total weight of the nail composition, as well as individual values or sub-ranges contained within.

v. Optional Abrasion or Scratch Resistance Additives

In certain instances, the nail compositions further include one or more additives that increase abrasion resistance or scratch resistance of the formed nail coating. The one or more additives to increase abrasion or scratch resistance can be present in a concentration ranging from about 0.0001 to 50 wt. % of the total weight of nail composition, as well as individual values or sub-ranges contained within. Without limitation, the one or more additives to increase abrasion or scratch resistance can be minerals, ceramics, silicas, silicones, nanoclays, comonomers such as isobornyl acrylate, allyl diglycol carbonate, polymer-based additives (such as poly(methyl methacrylate)), polysiloxanes, polyurethanes, polyolefins, MoS₂, graphite, oleic acid amide, aliphatic polyurethane acrylates, or a combination thereof.

### C. Properties of Formed Nail Coatings

The nail coatings formed from the nail compositions are coatings which result from a reaction of the active agent(s) and optional co-reactive monomers of the composition, such as a polymerization or other reaction. The properties of the nail coatings described below refer to the final nail coatings formed following the completion of such reaction processes.

The nail coatings can impart desirable properties on the nail surface, as compared to the same nail surface prior to formation of the nail coating thereon.

In some instances, the nail coating formed from the nail compositions described provides a sheen or gloss on the nail, as compared to the nail prior to formation of the nail coating thereon. In some instances, the sheen or gloss is increased by at least about 1 to 100%, as well as individual values or sub-ranges disclosed within. Sheen and gloss may be evaluated by visual means, such as by a person skilled in the art or a panel of such persons. Sheen and gloss can be observed visually by eye or with a microscope and compared to standard values. Sheen and gloss can be evaluated using, for instance, a glossmeter, such as a Glossgar portable glossmeter (Gardner Laboratories). Gloss can be dependent upon pigment concentrations, solids composition, and the composition of the nail coating. Gloss can be determined by projecting a beam of light at a fixed intensity and angle onto a surface and measuring the amount of reflected light at an equal but opposite angle. Measurements are typically made at an angle of 60 degrees. “Sheen” typically refers to a shine on a surface. “Gloss,” typically refers to a shine having a reflective quality of a surface that is smooth and that has a more intense shine, as compared to sheen.

In some instances, the nail coating strengthens, protects, provides scratch resistance, repairs, and/or hardens the nail of a subject, as compared to the nail prior to formation of the nail coating thereon. Hardness can be determined, for example, using a pencil hardness test or a Vickers or Knoop indenter. Additional testing systems can include, but are not limited to, the Sward Rocker and the Tukon Microhardness Tester. Other suitable techniques are described in the article by M. L. Schlossman, J. Soc. Cosmet. Chemists, 1981, 32, pages 43-52. In some instances, the strength, scratch resistance, or hardness of the nail is increased by at least about 1 to 100%, as well as individual values or sub-ranges disclosed within. In some instances, the strength, scratch resistance, or hardness of the nail is increased by at least about 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

In some instances, the nail coating increases resistance to wear and/or abrasion, and/or tearing, and/or robustness, and/or adhesion of subsequent coatings applied onto the nail coating, and optionally improves the look, and/or feel of the nail of a subject, as compared to the nail prior to application of the nail composition thereon. The increased resistance to wear and abrasion is assessed using a tribometer or by nail metrology (such as flexural, tensile, and/or tearing tests). In some instances, the wear/abrasion resistance, tearing resistance, robustness, look, or feel of the nail is increased by at least about 1 to 100%, as well as individual values or sub-ranges disclosed within. In some instances, the wear/abrasion resistance, tearing resistance, robustness, and/or improvement in look or feel of the nail is increased is increased by at least about 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Look, feel, and/or robustness can be assessed by visual or tactile inspection and may include a reduction of ridges and the filling in of defects present in the nail prior to formation of the nail coating thereon. In some instances, the improvement in look and/or feel may also be assessed by microscopic imaging, profilometry, and/or abrasion testing, which can compare the nail's surface quality before and after formation of the nail coating. In some instances, the adhesion of subsequent coatings is improved by at least about 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Adhesion can be measured with techniques well known in the art, such as a tack test or peel tests (e.g., at 90 degree or 180 degree angles).

In some instances, the nail coating formed on a nail can remain at least partially or fully intact when exposed to hot water (i.e., about 120° F.), cold water, or water containing salt and/or a surfactant. The nail coating can be characterized prior to and following exposure to the hot water (i.e., about 120° F.), cold water, or water containing salt and/or a surfactant to evaluate the coating's ability to remain at least partially or fully intact. The nail coating can be evaluated, for example, using microscopic imaging, profilometry, and/or abrasion testing.

In some instances, the nail coating formed from the nail compositions described herein exhibits self-healing and/or self-polishing properties. In other words, the nail coating exhibits the ability to automatically repair scratches and/or damage without any external intervention. Thus, the nail coating can in some instances fully restore or partially restore the nail coating to its as originally deposited state after being damaged by physical factors.

### III. Methods of Using the Nail Compositions

The nail compositions described herein can be used in various applications in the nail care industry. In one non-limiting instance, a method of forming a coating on a nail with the nail compositions includes the steps of:
(a) applying the nail composition to a subject's nail; and
(b) allowing the cosmetically acceptable carrier to evaporate to form a nail coating, optionally an initial coating, on the nail of the subject.

In some instances, the method further includes a step of exposing the nail composition to a stimulus, such as light or heat prior to, during, or following evaporation of the cosmetically acceptable carrier, where the stimulus is sufficient to induce a reaction of at least some of the active agent, for example, to undergo at least partial polymerization of at least a portion of the active agent to form a polymer, or coating thereof, containing disulfide or polysulfide bonds therein. In some instances, the light is selected from ultraviolet light, visible light, or sunlight. In some instances, the light has an exposure time on the nail composition which is in a time range from about 1 second to 10 minutes, 10 seconds to 5 minutes, or 15 seconds to 90 seconds, as well as individual values or sub-ranges disclosed within. In some other instances, the stimulus is heat and the nail composition is heated to about 35 to 100, 40 to 80, or 45 to 60° C., where the heat can have an exposure time on the nail composition in a time ranging from about 1 second to 10 minutes, 10 second to 5 minutes, or 15 seconds to 90 seconds, as well as individual values or sub-ranges disclosed within for any of the aforementioned temperature or time ranges.

In some instances, the subject is a human or animal and the nail is a fingernail or a toenail.

In some instances, the method forms a nail coating that provides a sheen or gloss on the nail, as compared to the nail prior to formation of the nail coating thereon. Such an improved sheen or gloss can in some instances be due to an increase in the refractive index of the coating compared to the natural nail plate or the generation of a smoother surface. In some instances, the method forms a nail coating that strengthens, protects, provides scratch resistance, repairs, and/or hardens the subject's nail, as compared to the nail prior to formation of the nail coating thereon. In some instances, the method forms a nail coating that increases resistance to wear and/or abrasion, and/or tearing, and/or robustness, and/or improves the look and/or feel of the subject's nail, as compared to the nail prior to application of the nail composition thereon. In some instances, the method forms a nail coating which provides an improvement in look, feel, and/or robustness, as assessed by visual or tactile inspection and may comprise a reduction of ridges and the filling in of defects present in the subject's nail prior to formation of the nail coating thereon.

The nail compositions can be applied to the subject's nail in any suitable form (such as in the form of a base coat, a color coat, a top coat, or a dip powder).

In some instances, the nail compositions can be used to form nail coatings that can be further used with other known nail polishes, lacquers, gel nail polishes, powder-based dip nails, etc., which are commercially available. In some instances, the method further includes a step of applying at least a second nail product, such as a nail polish, nail lacquer, a gel polish, or a dip powder, nail enhancement, artificial nail, or acrylic nail onto the nail coating. Such second nail products may be commercially available nail products. In some instances, such second nail products may be nail compositions such as those described herein. In such instances, the nail coating formed from the nail composition can act as a primer or promoter that improves adhesion of the second nail product on the nail plate.

### IV. Kits

The nail compositions described herein can be packaged together in any suitable combination as a kit useful for the disclosed methods. For example, the kits are provided for nail care applications, such as use as a nail strengthener, a nail primer, a nail protector, a nail polish, a nail gel, a nail extension, or a nail dip.

In one instance, a kit can include one or more of the nail compositions (such as in the form of a strengthener, a primer, a protector, a base coat, a color coat, a top coat, or a powder) described herein and at least one of the following:
(i) a nail trimmer, cuticle pusher, a nail buffer, and/or a nail file;
(ii) a UV lamp, LED lamp, visible light source, and/or infrared source;
(iii) a cuticle oil;
(iv) nail art (e.g., stickers, gems, freehand designs);
(v) a secondary nail product such as a gel nail system or a dip nail system; and/or
(vi) instructions for use.

In some instances, the kit contains the nail composition in the form of a nail strengthener or nail primer or nail protector, optionally in combination with one or more secondary nail products such as (i) a dip nail system that includes a bond coat, a base coat, a dip powder, an activator solution, and/or a top coat, which can be used for applying dip processed nails, or (ii) a gel nail system that includes a base coat, one or more color coats, and/or a top coat, which can be used for applying gel nails.

In some instances, the UV or LED lamp is a commercially available UV/LED nail lamp (such as a SUNUV SUN2C, LKES8) or a light-emitting diode source purchased from Thorlabs, Inc. In some instances, the UV or LED lamp provides a narrow emission wavelength which is centered around 365 nm, 385 nm, 405 nm, and 415 nm. In some instances, the narrow emission wavelength is centered in a range of about 365 to 415 nm.

### EXAMPLES

### Example 1: Examples of Nail Strengthener or Nail Primer Formulations Containing Lipoic Acid

### Materials and Methods

All formulations were prepared by dissolving each component in the listed solvent within a transparent 20 mL vial, dark brown 20 mL vial, or opaque black nail polish bottle. After mixing, the vial was gently shaken to dissolve all components, yielding a transparent yellow solution. These solutions were coated on fingernails and toenails by gentle painting with a nail-polish brush. After application, the coated nails were air dried for approximately 60-120 seconds. After drying, coated fingernails and toenails were exposed to different light sources for 30-120 seconds, including commercially available UV/LED nail lamps (SUNUV SUN2C, LKES8), and light-emitting diode sources purchased from Thorlabs, Inc. with narrow wavelength ranges centered around 365 nm, 385 nm, 405 nm, and 415 nm.

### Results

Observations for the nail coatings formed from each nail strengthener or nail primer composition after exposure to light are provided in Table 1. The composition of each polish formulation is reported as a weight percent (wt %) of each component relative to the total mass of the formulation as summarized in Table 1.

TABLE 1
Examples of nail strengthener compositions containing
lipoic acid and observations following curing on a nail

| | Lipoic | | Ethyl | | |
|---|---|---|---|---|---|
| | acid | BAPO | acetate | | |
| Entry | (wt %) | (wt %) | (wt %) | Other | Comments |
| 1 | 3 | 1 | 96 | — | Difficult to see |
| | | | | | film |
| 2 | 4 | 1 | 95 | — | Noticeable film at |
| | | | | | 4% lipoic acid |
| 3 | 7 | 2 | 91 | — | Nice and shiny |
| | | | | | film when coated |
| | | | | | on nail |
| 4 | 7 | 0 | 93 | — | Sample developed |
| | | | | | a foul odor |
| 5 | 15 | 3 | 82 | — | Coated well |
| | | | | | resulting in a shiny |
| | | | | | thick film |
| 6 | 20 | 3 | 77 | — | Sample is turbid |
| | | | | | due to limits of |
| | | | | | solubility |
| 7 | 15 | 1 | 84 | — | Sample developed |
| | | | | | a more pronounced |
| | | | | | odor on the nail |
| 8 | 15 | 3 | 78 | Vitamin E: 1%, | Nail feels nice |
| | | | | Jojoba oil: 1%, white | after with coating |
| | | | | eucalyptus oil: 2% | and has a pleasant |
| | | | | | smell. |
| 9 | 20 | 3 | 67 | Acetone: 10% | Sample still turbid |
| | | | | | with acetone |
| | | | | | cosolvent |
| 10 | 15 | 3 | 80 | n-butyl acrylate: 2% | Film was sticky |
| | | | | | after curing |
| 11 | 15 | 3 | 77 | n-butyl acrylate: 5% | Film was not as |
| | | | | | robust |
| 12 | 15 | 3 | 80 | Hydroxyethyl | Still had a slight |
| | | | | acrylate: 2% | odor after leaving |
| | | | | | in sun |
| 13 | 15 | 3 | 80 | Hydroxyethyl | Odor is nearly |
| | | | | acrylate: 2% | eliminated, but |
| | | | | | film is not as shiny |
| 14 | 15 | 3 | 81.6 | Trimethylolpropane | Robust film with |
| | | | | triacrylate: 0.4% | slight glossy finish |
| 15 | 15 | 3 | 80.5 | Trimethylolpropane | Robust film with a |
| | | | | triacrylate: 1.5% | glossy finish |
| 16 | 15 | 1 | 82.5 | Trimethylolpropane | Odor is eliminated |
| | | | | triacrylate: 1.5% | |
| 17 | 15 | 3 | 79 | Pentaerthyritol | Does not fully cure |
| | | | | tetrakis(3- | |
| | | | | mercaptopropionate): 3% | |
| 18 | 15 | 3 | 81.2 | Dipentaerythritol | Robust film with a |
| | | | | pentahexa-acrylate: 0.8% | slight glossy finish |
| 19 | 15 | 0 | 82 | Camphorquinone: 3% | Nice film but |
| | | | | | slightly tacky |
| 20 | 15 | 3 | 80.9 | Pentaerythritol | Robust film with a |
| | | | | tetraacrylate: 1.1% | glossy finish |
| 21 | 15 | 3 | 79.8 | Tripropylene glycol | Transparent film |
| | | | | diacrylate: 2.2% | but slightly tacky |

“—” denotes not present.

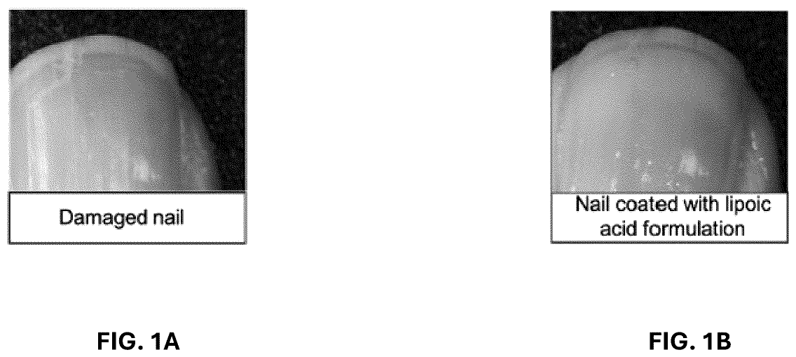

The comparison between damaged nails before and after application of a lipoic acid coating is demonstrated in FIGS. 1A and 1B, respectively. FIG. 1B shows the formulation of entry 5 of Table 1.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific instances of the invention described herein. Such equivalents are intended to be encompassed by the following claims.

1. A nail composition comprising:
an active agent having a molecular structure comprising at least one cyclic ring containing a disulfide or polysulfide bond; and
a cosmetically acceptable carrier;
wherein the active agent is capable of undergoing a reaction, optionally a polymerization reaction, when exposed to a stimulus;
wherein the concentration of active agent is sufficient to form a nail coating on a nail surface following application to the nail surface and exposure to the stimulus; and
wherein the nail coating is adherent to the nail surface.

2. (canceled)

3. The nail composition of claim 1, wherein the at least one cyclic ring containing a disulfide or polysulfide bond is a dithiolane.

4. The nail composition of claim 1, wherein the at least one cyclic ring containing a disulfide or polysulfide bond is a dithiane.

5. The nail composition of claim 1, wherein the at least one cyclic ring containing a disulfide or polysulfide bond is a dithiepane.

6. The nail composition of claim 1, wherein the active agent is α-lipoic acid, an ester of α-lipoic acid, an anhydride of α-lipoic acid, a salt of α-lipoic acid, an amide of α-lipoic acid, or a combination thereof.

7. The nail composition of claim 1, wherein the molecular structure of the active agent is according to Formula I: 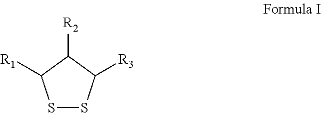
wherein at least one of R₁, R₂, and R₃ groups is a moiety independently selected from the group consisting of substituted or unsubstituted alkyl, substituted or unsubstituted alkenyl, substituted or unsubstituted alkynyl, substituted or unsubstituted aryl, carboxylic acid, —C(O)R^{a}, —C(O)OR^{a}, carboxylate, primary amide, secondary amide, —C(O)NR^{a}R^{b}, —NR^{a}R^{b}, alkyl, alkenyl, alkynyl, aryl, hydroxyl, anhydride (—C(O)—O—C(O)R^{a}), acrylate, methacrylate, acrylamide, styrene, substituted styrene, maleimide, epoxide, imide, lactone, urea (—NR^{a}—C(O)—NR^{a}R^{b}), ether (—OR^{c}), siloxy, thioether (—SR^{c}), ketone, thioketone, selenoketone, and combinations thereof, wherein R₁, R₂, and R₃ are hydrogen when they are not one of the aforementioned moieties;
wherein R^{a} and R^{b} are each independently selected from the group consisting of hydrogen, alkyl, alkenyl, alkynyl, aryl, cycloalkyl, aryl, heterocycloalkyl and heteroaryl; wherein each of R^{a} and R^{b} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof;
wherein R^{c} is selected from the group consisting of alkyl, alkenyl, alkynyl, aryl, cycloalkyl, aryl, heterocycloalkyl and heteroaryl; wherein R^{c} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

8. (canceled)

9. The nail composition of claim 7, wherein R₁ is —C(O)OR^{a} and R^{a} is selected from the group consisting of hydrogen, alkyl, alkenyl, alkynyl, aryl, cycloalkyl, aryl, heterocycloalkyl and heteroaryl; wherein the R^{a} group is optionally substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

10. The nail composition of claim 1, wherein the molecular structure of the active agent is according to Formula II: 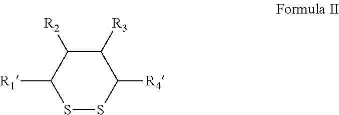
wherein at least one of R₁′, R₂′, R₃′, and R₄′ is a moiety independently selected from the group consisting of substituted or unsubstituted alkyl, substituted or unsubstituted alkenyl, substituted or unsubstituted alkynyl, substituted or unsubstituted aryl, carboxylic acid, —C(O)R^{a}, —C(O)OR^{a}, carboxylate, primary amide, secondary amide, —C(O)NR^{a}R^{b}, —NR^{a}R^{b}, substituted or unsubstituted alkyl, substituted or unsubstituted alkenyl, substituted or unsubstituted alkynyl, substituted or unsubstituted aryl, hydroxyl (—OH), anhydride (—C(O)—O—C(O)R^{a}), acrylate, methacrylate, acrylamide, styrene, substituted styrene, maleimide, epoxide, imide, lactone, urea (—NR^{a}—C(O)—NR^{a}R^{b}), ether (—OR^{c}), siloxy, thioether (—SR^{c}), ketone, thioketone, selenoketone, and combinations thereof, wherein the R₁′, R₂′, R₃′, and R₄′ groups are hydrogen when they are not one of the aforementioned moieties;
wherein R^{a} and R^{b} are each independently selected from the group consisting of hydrogen, alkyl, alkenyl, alkynyl, aryl, cycloalkyl, aryl, heterocycloalkyl and heteroaryl; wherein each of R^{a} and R^{b} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof; and
wherein R^{c} is selected from the group consisting of alkyl, alkenyl, alkynyl, aryl, cycloalkyl, aryl, heterocycloalkyl and heteroaryl; wherein R^{c} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

11. (canceled)

12. The nail composition of claim 1, wherein the molecular structure of the active agent is according to Formula III: 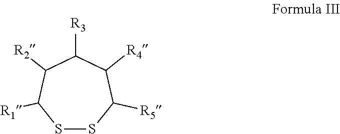
wherein at least one of R₁″, R₂″, R₃″, R₄″, and R₅″ is a moiety independently selected from the group consisting of substituted or unsubstituted alkyl, substituted or unsubstituted alkenyl, substituted or unsubstituted alkynyl, substituted or unsubstituted aryl, carboxylic acid, —C(O)R^{a}, —C(O)OR^{a}, carboxylate, primary amide, secondary amide, —C(O)NR^{a}R^{b}, —NR^{a}R^{b}, substituted or unsubstituted alkyl, substituted or unsubstituted alkenyl, substituted or unsubstituted alkynyl, substituted or unsubstituted aryl, hydroxyl, anhydride (—C(O)—O—C(O)R^{a}), acrylate, methacrylate, acrylamide, styrene, substituted styrene, maleimide, epoxide, imide, lactone, urea (—NR^{a}—C(O)—NR^{a}R^{b}), ether (—OR^{c}), siloxy, thioether (—SR^{c}), ketone, thioketone, selenoketone, and combinations thereof, wherein the R₁″, R₂″, R₃″, R₄″, and R₅″ groups are hydrogen when they are not one of the aforementioned moieties;
wherein R^{a} and R^{b} are each independently selected from the group consisting of hydrogen, alkyl, alkenyl, alkynyl, aryl, cycloalkyl, aryl, heterocycloalkyl and heteroaryl; wherein each of R^{a} and R^{b} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof; and
wherein R^{c} is selected from the group consisting of alkyl, alkenyl, alkynyl, aryl, cycloalkyl, aryl, heterocycloalkyl and heteroaryl; wherein R^{c} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

13. (canceled)

14. The nail composition of claim 1, wherein the molecular structure of the active agent is according to Formula IV: 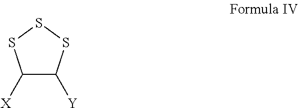
wherein at least one of X and Y is a moiety independently selected from the group consisting of substituted or unsubstituted alkyl, substituted or unsubstituted alkenyl, substituted or unsubstituted alkynyl, substituted or unsubstituted aryl, carboxylic acid, —C(O)R^{a}, —C(O)OR^{a}, carboxylate, primary amide, secondary amide, —C(O)NR^{a}R^{b}, —NR^{a}R^{b}, alkyl, alkenyl, alkynyl, aryl, hydroxyl, anhydride (—C(O)—O—C(O)R^{a}), acrylate, methacrylate, acrylamide, styrene, substituted styrene, maleimide, epoxide, imide, lactone, urea (—NR^{a}—C(O)—NR^{a}R^{b}), ether (—OR^{c}), siloxy, thioether (—SR^{c}), ketone, thioketone, selenoketone, and combinations thereof, wherein the X and Y groups are hydrogen when they are not one of the aforementioned moieties;
wherein R^{a} and R^{b} are each independently selected from the group consisting of hydrogen, alkyl, alkenyl, alkynyl, aryl, cycloalkyl, aryl, heterocycloalkyl and heteroaryl; wherein each of R^{a} and R^{b} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, heterocycloalkyl, cycloalkyl, heteroaryl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof; and
wherein R^{c} is selected from the group consisting of alkyl, alkenyl, alkynyl, aryl, cycloalkyl, aryl, heterocycloalkyl and heteroaryl; wherein R^{c} is optionally independently substituted with one or more substituents selected from the group consisting of halogen, hydroxyl, cyano, isocyano, cyanato, isocyanato, nitro, amino, alkylamino, dialkylamino, ammonium, tetra-substituted ammonium, alkyl, aryl, acyl, heterocycloalkyl, cycloalkyl, haloalkyl, hydroxyalkyl, carboxy, alkoxy, aryloxy, alkoxycarbonyl, aminocarbonyl, alkylaminocarbonyl, siloxy, dialkylaminocarbonyl, and combinations thereof.

15. (canceled)

16. The nail composition of claim 1, wherein the active agent is a multifunctional compound comprising one or more moieties, wherein each of the one or more moieties includes at least one cyclic ring containing a disulfide or polysulfide bond.

17. The nail composition of claim 16, wherein the one or more moieties comprise a structure according to any one of Formulae I-IV.

18. The nail composition of claim 1, wherein the active agent is selected from the group consisting of 1,2-dithiolane, asparagusic acid, 1,2,3-trithiolane, S₈, 1,2-dithiane, 1,2-dithepane, and combinations thereof.

19. The nail composition of claim 1, wherein the nail composition further comprises one or more co-reactive monomers capable of undergoing copolymerization or condensation with the active agent.

20-22. (canceled)

23. The nail composition of claim 1, wherein the active agent is at least partially polymerized and forms a polymer or copolymer comprising disulfide or polysulfide bonds.

24. The nail composition of claim 1, wherein the active agent is attached to a polymer backbone selected from the group consisting of polysiloxane, polyester, polyether, polyacrylate, polymethacrylate, polystyrene, polyolefin, polyurethane, polycarbonate, polyamide, polyurea, polypeptide, polypeptoid, and polyamine polymer backbones.

25. The nail composition of claim 23, wherein the polymer is poly(lipoic acid), poly(alkyl lipoate), poly(lipoamide), a poly(disulfide) formed by condensation polymerization, a poly(disulfide) formed by oxidative coupling, a poly(disulfide) formed by ring opening polymerization, a poly(disulfide) formed by autopolymerization, a poly(disulfide) formed by cationic polymerization, a poly(disulfide) formed by anionic polymerization, a poly(disulfide) formed by radical polymerization, or copolymers thereof.

26. The nail composition of claim 1, wherein the active agent is present in a concentration ranging from about 1 to 100 wt. %, 3 to 100 wt. %, 5 to 100 wt. %, 10 to 100 wt. %, 15 to 100 wt. %, 20 to 100 wt. %, or 25 to 100 wt. % of the total weight of the nail composition in the absence of the cosmetically acceptable carrier.

27-30. (canceled)

31. The nail composition of claim 1, wherein the nail composition further comprises one or more cosmetically acceptable excipients.

32. (canceled)

33. The nail composition of claim 1, wherein the nail composition further comprises one or more photochemical and/or thermal initiators.

34-37. (canceled)

38. The nail composition of claim 1, wherein the nail composition further comprises:
at least one stabilizing agent which reduces changes in odor, appearance, composition and/or viscosity over time compared the same composition in the absence of the stabilizing agent(s) for a period of time ranging from at least about 3 to 24 months or 6 to 12 months;
a thiol capping agent, which reduces changes in odor, appearance, composition and/or viscosity over time compared the same composition in the absence of the capping agent(s) for a period of at least about 3 to 24 or 6 to 12 months;
one or more ultraviolet absorbing agents; and/or
one or more additives to increase abrasion or scratch resistance.

39-47. (canceled)

48. The nail composition of claim 23, wherein the nail composition further comprises a second monomer or co-monomer which does not undergo co-polymerization with the active agent;
wherein the second monomer or co-monomer forms a second polymer or polymeric network that is independent from the polymer formed from the active agent.

49. (canceled)

50. The nail composition of claim 38, wherein the one or more additives to increase abrasion or scratch resistance are selected from the group consisting of minerals, ceramics, silicas, silicones, nanoclays, comonomers, polymer-based additives, polysiloxanes, polyurethanes, polyolefins, MoS₂, graphite, oleic acid amide, aliphatic polyurethane acrylates), and combinations thereof.

51. The nail composition of claim 1, wherein the nail composition is a nail strengthener, a nail primer, a nail protector, a nail polish, a nail lacquer, a nail gel, or a nail dip.

52. (canceled)

53. A method of forming a coating on a nail comprising:
(a) applying the nail composition of claim 1 to a subject's nail; and
(b) allowing the cosmetically acceptable carrier to evaporate to form a nail coating, optionally an initial coating, on the nail of the subject.

54. The method of claim 53, comprising a step of exposing the nail composition to a stimulus prior to, during, or following evaporation of the cosmetically acceptable carrier;
wherein the stimulus is sufficient to induce a reaction of at least some of the active agent, for example, to undergo at least partial polymerization of at least a portion of the active agent to form a polymer or coating containing disulfide or polysulfide bonds therein.

55. The method of claim 54, wherein the stimulus is light which is selected from ultraviolet light, visible light, or sunlight; wherein the light has an exposure time on the nail composition which is in a time range from about 1 second to 10 minutes, 10 seconds to 5 minutes, or 15 seconds to 90 seconds.

56. The method of claim 54, wherein the stimulus is heat and the nail composition is heated to about 35 to 100, 40 to 80, or 45 to 60° C.; wherein the heat has an exposure time on the nail composition which is in a time ranging from about 1 second to 10 minutes, 10 second to 5 minutes, or 15 seconds to 90 seconds.

57-59. (canceled)

60. The method of claim 53, wherein the nail coating provides a sheen or gloss on the nail, as compared to the nail prior to formation of the nail coating thereon.

61. The method of claim 53, wherein the nail coating strengthens, protects, provides scratch resistance, repairs and/or hardens the subject's nail, as compared to the nail prior to formation of the nail coating thereon.

62. (canceled)

63. The method of claim 53, wherein the nail coating increases resistance to wear and/or abrasion, and/or tearing, and/or robustness, and/or adhesion of subsequent coatings applied onto the nail coating, and optionally improves the look and/or feel of the nail of a subject, as compared to the nail prior to application of the nail composition thereon.

64-69. (canceled)

70. A kit comprising the nail formulation of claim 1.
